SEQUENCE LISTING (28 sequences)

SEQ ID NO: 1 - DNA, 1471 bp, Homo sapiens
  Oligonucelotide sequence (1)..(1471)
tggatccttttgtggtccttgtgctctgtctctcatgtttgcttctcctttcaatctgagacagagctctgggagaggaaaactccctcctggccccactcctctcccagtgattggaaatatcctacagatagatattaaggatgtcagcaaatccttaaccaatctctcaaaaatctatggccctgtgttcactctgtattttggcctggaacgcatggtggtgctgcatggatatgaagtggtgaaggaagccctgattgatcttggagaggagttttctggaagaggccatttcccactggctgaaagagctaacagaggatttggaatcgttttcagcaatggaaagagatggaaggagatccggcgtttctccctcatgacgctgcggaattttgggatggggaagaggagcattgaggaccgtgttcaagaggaagcccgctgccttgtggaggagttgagaaaaaccaaggcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctggatccagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctga

SEQ ID NO: 2 - DNA, 26 bp, Artificial
  Artifical Sequence (Forward Primer Exon 4)
atccccaactattctcaccctttcta

SEQ ID NO: 3 - DNA, 23 bp, Artificial
  Artifical Sequence(Reverse Primer Exon 4)
gatattcaccccatggctgtcta

SEQ ID NO: 4 - DNA, 23 bp, Artificial
  Artifical Oligonucelotide (Forward Primer Exon 5)
gatgtctcagttgggattctagc

SEQ ID NO: 5 - DNA, 28 bp, Artificial
  Artifical oligonucelotide (Reverse Primer Exon 5)
atcacaaatacgcaagcagtcacataac

SEQ ID NO: 6 - DNA, 25 bp, Artificial
  Artifical Oligonucelotide (Forward primer Exon 1)
ggacaaagtctcctaatcttcgata

SEQ ID NO: 7 - DNA, 28 bp, Artificial
  Reverse Primer Exon 1
acaatgatctcttgtaacattgtacctc

SEQ ID NO: 8 - DNA, 24 bp, Artificial
  Artifical Sequence (Forward Primer Exon 2)
gagcctgtgtgactgaataaaagc

SEQ ID NO: 9 - DNA, 23 bp, Artificial
  Artifical Sequence (Reverse Primer Exon 2)
atttccaccatgtccacttgtca

SEQ ID NO: 10 - DNA, 20 bp, Artificial
  Artifical Sequence (Forward Primer Exon 9)
tgtgttggggactgatgatg

SEQ ID NO: 11 - DNA, 23 bp, Artificial
  Synthetic Oligonucleotide (Reverse Primer exon 9)
ggaggctgaatgttcactagatc

SEQ ID NO: 12 - DNA, 21 bp, Artificial
  Oligonucleotide Sequence (SNaPshot Primer 1)
ccactttcatcctgggctgtg

SEQ ID NO: 13 - DNA, 23 bp, Artificial
  Oligonucelotide Primer (SNaPshot Primer 2)
cccactatcattgattatttccc

SEQ ID NO: 14 - DNA, 21 bp, Artificial
  Oligonucleotide Primer (SNaPshot Primer 3)
ggagcagatcacattgcaggg

SEQ ID NO: 15 - DNA, 20 bp, Artificial
  SNaPshot Primer 4
gccctgattgatcttggaga

SEQ ID NO: 16 - DNA, 19 bp, Artificial
  Oligonucleotide primer (SNaPshot Primer 5)
ggattgtaagcaccccctg

SEQ ID NO: 17 - DNA, 25 bp, Artificial
  Oligonucleotide Primer (SNaPshot Primer 6)
gccaggccctctcccacacaaatcc

SEQ ID NO: 18 - DNA, 750 bp, Artificial
  PCR product corresponding to Exon 1
ggacaaagtctcctaatcttcgatataggattgacatactttttaaatatacaaggcatagaatatggccatttccgttaaatcataaattcccaactggttattaatctaagaattcagaattttaagtaattgtttttgcatcagattgtttacttcagtgctctcaattatgacggtgcattggaaccacttgggttaacatttttttgtttttattaccaatacctaggcttcaacctagtacaatgaaaccagaatgtacagagtgggcactgggacgaaggagaacaagaccaaaggacattttatttttatctctatcagtgggtcaaagtcctttcagaaggagcatatagtgggcctaggtgattggccactttatccatcaaagaggcacacacacttaattagcatggagtgttataaaaagcttggagtgcaagctcacggttgtcttaacaagaggagaaggcttcaatggatccttttgtggtccttgtgctctgtctctcatgtttgcttctcctttcaatctggagacagagctctgggagaggaaaactccctcctggccctactcctctcccagtgattggaaatatcctacagatagatattaaggatgtcagcaaatccttaaccaatgtaagtatgctccttcagtggcttgcaaaaggtaagtaaattcacctgtattttttaaataaagtatatccctagaggtacaatgttacaagagatcattgt

SEQ ID NO: 19 - DNA, 984 bp, Artificial
  PCR product corresponding to exon 2
gagcctgtgtgactgaataaaagcatacaaatacaatgaaaatatgaatctaagtcaggcttagtaaatggacaaaacagtgacttcatttgctgttaactgtatctccttttctagctctcaaaaatctatggccctgtgttcactctgtattttggcctggaacgcatggtggtgctgcatggatatgaagtggtgaaggaagccctgattgatcttggagaggagttttctggaagaggccatttcccactggctgaaagagctaacagaggatttggtaggtgtgcaagtgcctgtttcagcatctgtcttggggatggggaggatggaaaacagactagcagagcttctcgggcagagcttggcccatccacatggctgcccagtgtcagcttcctctttcttgcctgggatctccctcctagtttcgtttctcttcctgttaggaatcgttttcagcaatggaaagagatggaaggagatccggcgtttctccctcatgacgctgcggaattttgggatggggaagaggagcattgaggaccgtgttcaagaggaagcccgctgccttgtggaggagttgagaaaaaccaagggtgggtgaacatactctctatcactgacctttctggactgctctcctctctactgacattcttggaaacatttcaggggtggccagatcttttatttggagtcctggttgttagctcatgtgaagcagggtttgaagctgagagccaagggaatttgcacatgtttgtgctgtgtgtgtacaggcatgattgtgcatacagtgtgggtataaaagttcatttaatcctatgttctcctgaactttgcttctttgttttcaaataagaaatgatgaatatagattttgagttcattttttgaaagagttaaagagcagtgtttttcccattatctattccagaacatgtcaccagagaatacttgacaagtggacatggtggaaat

SEQ ID NO: 20 - DNA, 684 bp, Artificial
  PCR product corresponding to exon 4
atccccaactattctcaccctttctatctcagtgctttgttgtctacagactttgcagactgatgtgattccctctgaaacttgaattatttggtttctaaaaaagtctctttttttctttccaaagtaaaagacaaataggccgggaatgtaaatttagcatttgagcaaccattatttaaccagctaggctgtaattgttaattcgagattaatgtaaaagtgatgtgttgattttatgcatgccaaactcttttttgcttttaagggaattcataggtaagatattacttaaaatttctaaactattattatctgttaacaaatatgaagtgttttatatctaatgtttactcatattttaaaattgtttccaatcatttagcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctggatccaggtaaggccaagttttttgcttcctgagaaaccacttacagtctttttttctgggaaatccaaaattctatattgaccaagccctgaagtacatttttgaatactacagtcttgcctagacagccatggggtgaatatc

SEQ ID NO: 21 - DNA, 902 bp, Artificial
  PCR product corresponding to exon 5
gatgtctcagttgggattctagctgatgagacagctggttaggaatgaaaaaattattgtttttttggtgtatgaaccataaacagacatcacacttttaccctgtgctgaattggcatgttttatactctgcctaaataataattgtatgattttacagaagtcatttaactgctctggtgcacagttggaatttgaagtatctttgagcccctcccacttctaaaatactaattcaatttcagaggctgcttgatagaaatcaatatagcagggactatctttgtagtatcaatcaggttgtgcaaactcttttaacctatgctatcatctccaaaatgttaatgtagtaattcataccatcttatatttcaagattgtagagaagaattgttgtaaaaagtaagagaattaatataaagatgcttttatactatcaaaagcaggtataagtctaggaaatgattatcatctttgattctcttgtcagaattttctttctcaaatcttgtataatcagagaattactacacatgtacaataaaaatttccccatcaagatatacaatatattttatttatatttatagttttaaattacaaccagagcttggcatattgtatctatacctttattaaatgcttttaatttaataaattattgttttctcttagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggtaaaatgttaacaaaagcttagttatgtgactgcttgcgtatttgtgat

SEQ ID NO: 22 - DNA, 989 bp, Artificial
  PCR product corresponding to exon 9
tgtgttggggactgatgatgagtggctatgcctagagttgagagtaggggaggtgaagagtgtaaaatgtgatacggatcatcagactggaaacagaatgtgagggtccaggtcaagctatttggatcttatcccataggacagacagggaagccatttaaagttttaaagtgagggggtaacatgtttagacatgtgattctgaaaatacttagcgggaaaaaaatctttggctgcatactgagccagaaatacaaagggaaatgccgaatgtgagcctcctcccctaagcccttctcagttcaacccactggacaggtaatgtatgtttctcaagaaagattttaatgaattgcttggcatgtagctgacagtcaataaacatttgttgaatacatggtgcttgataagatctgaagataggtgaagagtaagcatgtccattcattgtttagttgcctatccatccattcatccattaatccttccacccatccatcctttcattcatgcattcacccaaccacccatctatctactcatccctcctatgattcaccgaacagttcttgcatattctgtctgtgccagttatagagacagtgtttgtcactctcacagttacacatgaggagtaacttctccctatgtttgttattttcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctgaagaagcacagatggtctggctgctcctgtgctgtccctgcagctctctttcctctggtccaaatttcactatctgtgatgcttcttctgacccgtcatctcacattttcccttcccccaagatctagtgaacattcagcctcc

SEQ ID NO: 23 - protein, 490 aa, Homo sapiens
  Protein Sequence (1)..(490): Protein Sequence
MDPFVVLVLCLSCLLLLSIWRQSSGRGKLPPGPTPLPVIGNILQIDIKDVSKSLTNLSKIYGPVFTLYFGLERMVVLHGYEVVKEALIDLGEEFSGRGHFPLAERANRGFGIVFSNGKRWKEIRRFSLMTLRNFGMGKRSIEDRVQEEARCLVEELRKTKASPCDPTFILGCVPCNVICSIIFQKRFDYKDQQFLNLMEKLNENIRIVSTPWIQICNNFPTIIDYFPGTHNKLLKNLAFMESDILEKVKEHQESMDINNPRDFIDCFLIKMEKEKQNQQSEFTIENLVITAADLLGAGTETTSTTLRYALLLLLKHPEVTAKVQEEIERVVGRNRSPCMQDRGHMPYTDAVVHEVQRYIDLIPTSLPHAVTCDVKFRNYLIPKGTTILTSLTSVLHDNKEFPNPEMFDPRHFLDEGGNFKKSNYFMPFSAGKRICVGEGLARMELFLFLTFILQNFNLKSLIDPKDLDTTPVVNGFASVPPFYQLCFIPV

SEQ ID NO: 24 - DNA, 1471 bp, Homo sapiens; identical to SEQ ID NO: 1
  Nucleotide sequence (1)..(1471): Nucleotide sequence

SEQ ID NO: 25 - DNA, 189 bp, Artificial
  PCR product corresponding to exon 7
gctaaagtccaggaagagattgaacgtgtcgttggcagaaaccggagcccctgcatgcaggacaggggccacatgccctacacagatgctgtggtgcacgaggtccagagatacatcgactcatccccaccagcctgccccatgcagtgacctgtgacgttaaattcagaaactacctcattcccaagg

SEQ ID NO: 26 - DNA, 30 bp, Artificial
  Oligonucleotide primer
acgttggatgctgtaagtggtttctcagga

SEQ ID NO: 27 - DNA, 30 bp, Artificial
  Oligonucleotide primer
acgttggatgccaatcatttagcttcaccc

SEQ ID NO: 28 - DNA, 17 bp, Artificial
  Oligonucleotide primer
agatcacattgcaggga

(19) United States
(12) Patent Application Publication
(10) Pub. No.: US 2011/0245492 A1
(43) Pub. Date: Oct. 6, 2011

(54) NOVEL ALLELIC VARIANT OF CYP2C19 ASSOCIATED WITH DRUG METABOLISM

(75) Inventors: Brahmachari Samir KUMAR, New Delhi (IN); Kukreti Ritushree, New Delhi (IN); Mukerji Mitali, New Delhi (IN); Martis Suparna, Mumbai (IN); Fernandes Ravina, Mumbai (IN); Sharma Nitin, Mumbai (IN)

(73) Assignees: COUNCIL OF SCIENTIFIC AND INDUSTRIAL RESEARCH;; NICHOLAS PIRAMAL INDIA LIMITED

(21) Appl. No.: 13/108,337

(22) Filed: May 16, 2011

Related U.S. Application Data
(62) Division of application No. 11/183,327, filed on Jul. 15, 2005, now abandoned.

(30) Foreign Application Priority Data
Jul. 15, 2004 (IN) 1295/DEL/2004

(51) Int. Cl. C07D 243/24; C40B 20/08; C12Q 1/68; C07D 401/12; C07D 307/87; C07C 279/18

(52) U.S. Cl. 540/514; 506/6; 435/6.11; 546/273.7; 549/467; 564/234

(57) ABSTRACT

The invention provides methods, PCR primers and sequence determination oligonucleotides for determining a human's capacity to metabolise a substrate of the CYP2C19 enzyme using genetic analysis.

### CROSS-REFERENCE TO RELATED APPLICATIONS

This application is a divisional of U.S. patent application Ser. No. 11/183,327 filed Jul. 15, 2005, which claimed the benefit of the following Indian Patent Application: 1295/DEL/2004 filed Jul. 15, 2004, the contents of which are incorporated herein by reference.

### FIELD OF THE INVENTION

The present invention relates to a novel allelic variant of CYP2C19 gene. More particularly, it relates to a method of detection of a novel allelic variant comprising of certain polymorphisms in the exons of the gene encoding cytochrome P450 2C19, also known as CYP2C19, S-mephenyloin-4′-hydroxylase, to predict variations in an individual's ability to metabolise certain drugs.

### BACKGROUND OF THE INVENTION

It is well recognized that different patients respond in different ways to the same medication. The existence of large population differences with small intrapatient variability indicates the role of inheritance in determining drug response. Although many nongenetic factors influence the effects of medications including age, nutritional status, renal and liver function and concomitant therapy, it is estimated that genetics can account for 20 to 95 percent of variability in drug disposition and effects. There are numerous examples highlighting inter individual differences in drug response due to sequence variants in genes encoding drug metabolizing enzymes, drug transporters or drug targets.

Following the initial sequencing of the human genome, more than 1.4 million single nucleotide polymorphisms were identified. Through pharmacogenomic studies some of these SNPs have already been associated with substantial changes in the metabolism or effects of medications and some are being used to predict clinical response. Thus providing a powerful platform for optimizing drug therapy on the basis of each patient's genetic constitution.

In pharmacogenetic studies, the genotype of polymorphic alleles encoding one or more drug-metabolizing enzymes is determined and linked to an individual's drug metabolism phenotype. Determination of these genetic polymorphisms would be of clinical value in predicting adverse or inadequate response to certain therapeutic agents and in predicting increased risk of environmental or occupational exposure-linked disease.¹⁻³ The goal of pharmacogenetics is to examine the genome of an individual patient and design a drug treatment strategy tailored to that patient's particular drug metabolism profile. Assays and other methods by which drug-metabolizing polymorphisms in an individual's genome are determined thus have utility in the field of pharmacogenetics. Preferably, such assays are accurate (e.g., few false positives or negatives) and performed quickly.

Xenobiotics are pharmacologically, endocrinologically or toxicologically active substances foreign to a biological system. Most xenobiotics, including pharmacologically active molecules are lipophillic and remain un-ionized or partly ionized at physiological pH. The primary purpose of xenobiotic metabolism is to enzymatically convert a lipid-soluble xenobiotic into polar, water soluble and excretable metabolites that can be eliminated. It can also convert prodrugs into therapeutically active compounds, and it may even result in the formation of toxic metabolites. Pharmacologically drug (xenobiotic) metabolism pathways are classified as either Phase I reactions which are functionalization reactions (i-e oxidation, reduction and hydrolysis) in which a derivatizable group is added to the original molecule. Functionalization prepares the drug for further metabolism in Phase II reactions. Phase II metabolism involves enzyme catalysed conjugation of xenobiotics with groups such as glucoronic acid, sulphate and glutathione. The effect of these reactions is to greatly increase water solubility, aiding excretion in urine or bile.

In humans the cytochrome P-450 enzymes, a superfamily of microsomal drug-metabolising enzymes, play a central role in Phase I drug metabolism where they are of critical importance to two of the most significant problems in clinical pharmacology: drug interactions and interindividual variability in drug metabolism. More than seventy five cytochrome P450 genes, including nineteen psedogenes have been identified in humans, indicating the diversity of cytochrome P450 family. Members of three CYP gene families, CYP1, CYP2 and CYP3 are responsible for the majority of drug metabolism. The human CYPs which are of greatest clinical relevance for the metabolism of drugs and other xenobiotics are CYP1A2, CYP2D6, CYP2C9, CYP2C19, CYP2E1 and CYP3A4. The liver is the major site of activity of these enzymes, however CYPs are also expressed in other tissues.

The CYP2C19 enzyme is responsible for metabolism of a wide range of substrates like proton pump inhibitors omeprazole, lansoprazole and pentaprazole; antimalarial drugs such as proguanil; antidepressants such as citalopram; the benzodiazepines diazepam and flunitrazepam. In addition CYP2C19 acts in sidechain oxidation of propranolol and in demethylation of imipramine.

CYP2C19 is a polymorphic enzyme, that is, more than one form of the enzyme is present within the human population. These polymorphic variants impact the CYP2C19 enzyme activity by altering the rate at which substrate drugs are removed from the body and consequently wide variations in responses to such drugs including susceptibility to side effects have been observed.

Through probe drug phenotyping of S-mephenyloin or omeprazole, an individual's capacity to metabolise these CYP2C19 specific substrates can be assessed. Individuals who have normal metabolic activity are called fast or extensive metabolisers (EMs). Those who are deficient in their ability to metabolize the probe drug are characterized as slow or poor metabolisers (PMs). These poor metabolisers retain the CYP2C19 substrate for a relatively longer period of time and consequently are susceptible to toxicity and side effects at dosages well tolerated by normal or extensive metabolisers. Intermediate metabolisers (IMs) show metabolic activity between those of PMs and EMs. Ultrarapid extensive metabolisers (UEMs) clear the CYP2C19 substrate from their bodies faster than EMs and require higher dosages than normal metabolisers to achieve the therapeutic effect.

The existence of more than one form of the CYP2C19 enzyme is caused by polymorphisms in the gene which encodes the CYP2C19 enzyme (the gene being denoted in italics, as CYP2C19, (GenBank Ref: E10866 herein designated as SEQ ID NO:1). Currently more than ten polymorphisms have been reported (see http://www.imm.ki.se/CYPalleles for listing). (Lamba J K et al 2000, Lamba J K et al 1998, Nowak et al 1998, Adithan C. et al 2003, Lamba J K et al. 2001, Badyal D K and Dadhuch, A P 2001, Gaedigk, A 2000). All polymorphism positions correspond to the cDNA GenBank Ref: E10866 (SEQ ID NO. 1). The genetic sequence encoding an active enzyme is designated CYP2C19*1 and is commonly referred to as the wild type gene. The distribution and frequencies of CYP2C19 polymorphisms differ widely among different populations and ethnic groups, and association studies have established concomitant differences in CYP2C19 activity and responses to drugs which are CYP2C19 substrates (see http://www.imm.ki.se/CYPalleles for listing). About 3% of Caucasians have been found to be PMs of S-mephenyloin with very little variation noted between studies⁴. By contrast, several independent studies have shown a much higher incidence of PMs in Orientals, up to 18-23% in Japanese; 15-17% in Chinese; 12-16% in Koreans. In Africans, PM frequencies vary between 4 and 7%. The PM condition is inherited as an autosomal recessive trait⁴. The best characterized defect CYP2C19 polymorphisms responsible for the PM phenotype are: a single base pair substitution in exon 5 at position 681(G→A) of the coding sequence (GenBank Acc No. E10866) designated CYP2C19*2 alleles. The CYP2C19*2 allele is further subdivided into CYP2C19*2A and CYP2C19*2B alleles⁶. CYP2C19*2B allele has an additional polymorphism at position 276 (G→C) of the coding sequence. In Caucasians, CYP2C19*2A comprise 85% of poor metaboliser CYP2C19*2 allele while CYP2C19*2B accounts for the remaining 15%. A second single base pair change in exon 4 at position 636 (G→A) of the coding sequence, is designated CYP2C19*3 allele⁴. The change in CYP2C19*2 creates an aberrant splice site, resulting in truncated, inactive protein. This polymorphism accounts for 75% of the defective alleles in orientals and 93% in Caucasians (see http://www.imm.ki.se/CYPalleles for listing). The other well characterized detrimental allele CYP2C19*3 discovered in Japanese PMs, results in a stop codon and consequently an inactive protein. This allele accounts for approximately 25% of all inactive forms in orientals, being by converse extremely rare in non-oriental populations. Heterozygous carriers of one deficient allele of CYP2C19*2 and CYP2C19*3 and the other CYP2C19*1 allele are intermediate metabolisers^{10,11,14}.

In Indian population, subjects mainly from North India showed PM frequency of 11%. The CYP2C19*2 allele accounts for 57% of the defective alleles in poor metabolisers. Further in North Indians, the CYP2C19*3 allele was not detected⁷. Based on the foregoing, it is apparent that genetic polymorphisms of P450 enzymes result in phenotypically distinct subpopulations that differ in their ability to perform particular drug biotransformation reactions. These phenotypic distinctions have important implications in pharmacogenomics in terms of prescription of drugs and clinical trials. There is a definite need to identify individuals who are either deficient in CYP2C19 to prevent intolerable side effects. Alternatively, a drug that is effective in most humans may be ineffective in a particular subpopulation because of lack of CYP2C19 required for conversion of the drug to a metabolically active form. The novel polymorphism C518T of the present invention is unique as it has been found in heterozygous and homozygous recessive carriers and can function both as intermediate and poor metaboliser. This polymorphism could be functionally important and got selected out.

U.S. Pat. No. 5,786,191 discloses methods of screening for drugs metabolized by CYP2C19 using the CYP2C19 polypeptide. U.S. Pat. No. 5,912,120 and related WO 95/30766 disclose methods of diagnosis of a deficiency in CYP2C19 activity caused by the CYP2C19*2 and CYP2C19*3 polymorphisms. WO 00/12757 discloses a primer extension assay and kit for detection of single nucleotide polymorphisms in cytochrome P 450 isoforms, including the CYP2C19*2 and CYP2C19*3 polymorphisms. US Pat. Application No. 20030059774 discloses methods of detecting CYP2C19 UEMs through genetic analysis of three polymorphisms of the 5′ flanking region of the CYP2C19 gene. Although it is known that use of omeprazole as a probe drug reveals CYP2C19 IMs, very little characterization of the genetics of these individuals exists. The broad overlap between homozygous EM and heterozygous carriers of one deficient allele (IM) may lead to the wrong conclusion that this differentiation is not important. Comparing the functional differences between PMs, EMs and IMs, the pharmacokinetic parameters of the IMs can be close to PMs in some cases and close to EMs in others. Hence IMs need to be separately analysed in drug development and deserve careful consideration in genotype-based dose adjustments. From a pharmacogenomics point of view doses adjusted for the EM/IM difference will result in more appropriate therapy. There is a need to identify new polymorphisms and haplotypes defining IMs to increase sensitivity of testing through genotype-based dose adjustments. There is also a need for methods for diagnosing individuals exhibiting intermediate CYP2C19 activity. Further there is also a requirement to detect novel alleles for poor metabolisers to increase sensitivity of pharmacogenomic testing. The present invention fulfills these needs and other

### OBJECTS OF THE INVENTION

Main object of the invention is to provide novel allelic variant.

Another object of the invention is to provide a method for detection of the novel allelic variant of the CYP2C19 gene.

Still another object is to provide a diagnostic kit for prediction of CYP2C19 mediated drug response.

### SUMMARY OF THE INVENTION

The present inventors have concluded that in Indian population individuals who are homozygous or heterozygous for certain haplotypes consisting of polymorphic sites in exons 4 and 5 of the CYP2C19 gene, are likely to exhibit characteristic metabolic ratios for substrates of CYP2C19. Using this information, the capacity of individuals to metabolise drugs which are substrates of the CYP2C19 enzyme may be predicted by genotyping those polymorphisms. The invention also provides polymorphisms which obviates screening of certain CYP2C19 alleles in Indian population.

### DETAILED DESCRIPTION OF THE INVENTION

The present invention provides a novel allelic variant of CYP2C19 gene and its detection which will be useful in identifying intermediate and poor metabolisers of a substrate of CYP2C19. The invention also provides polymorphisms of CYP2C19 gene for detecting poor metabolisers in the Indian population. The invention also provides a novel diagnostic kit useful for rapid and economical pharmacogenomics based diagnostics for the Indian population.

Accordingly, the present invention provides a novel allelic variant CYP2C19*2C of CYP2C19 gene encoding the drug metabolizing enzyme CYP2C19 comprising SEQ ID No.24.

In an embodiment of the present invention, the length of the said variant comprises 1473 nucleic acid base pairs.

In another embodiment of the present invention, the novel polymorphism C/T is at position 518 of the SEQ ID No.24.

Further, the present invention also provides a novel polymorph of the CYP2C19 drug metabolizing enzyme comprising amino acid sequence of SEQ ID No. 23.

In an embodiment of the present invention, the length of the said polymorph comprises 490 amino acid residues.

In another embodiment of the present invention, novel polymorphism A/V is at position 173 of the SEQ ID No.23.

Still the present invention also provides a set of novel PCR primers useful for detection of the novel polymorphism of C518T comprising SEQ ID No. 2 and SEQ ID No. 3, wherein:

forward primer (SEQ ID No. 2):
5′ ATCCCCAACTATTCTCACCCTTTCTA 3′,
reverse primer (SEQ ID No. 3):
5′ GATATTCACCCCATGGCTGTCTA 3′.

In an embodiment of the present invention, a set of novel sequence determination oligonucleotide (SNap Short Primers) useful for detection of the novel polymorphism of C518T comprising SEQ ID No. 12 and SEQ ID No. 14, wherein:
forward primer (SEQ ID No. 12): 5′ CCACTTTCATCCTGGGCTGTG3′
reverse primer (SEQ ID No.14): 5′GGAGCAGATCACATTGCAGGGA3′.

Further, the present invention also provides a method for predicting the capacity of a drug dose to metabolize a substrate of a CYP2C19 enzyme in a human subject, wherein the said method comprising the steps of (a) preparing the nucleic acid template for identifying the polymorphism at positions 518 of SEQ ID NO: 2 and 3 comprising:

(i) isolating double stranded DNA from the subject;
(ii) PCR amplification of the DNA wherein the amplified DNA encodes exon 4 of CYP2C19 gene present on each homologous chromosome 10 of the subject as set forth in SEQ ID NO. 2 and 3 respectively;
(iii) purifying said amplified DNA by polyethylene glycol precipitation;
(b) detecting polymorphism using extension primers (Snap short primers) having SEQ ID No. 12 and 14;
(c) relating the labeled nucleic acid to the identity of the said polymorphism in the subject and validating the polymorphism by high throughput mass array detector using PCR primers comprising SEQ ID No. 26 and SEQ ID No. 27 and extension primer having SEQ ID 28, wherein:

forward primer (SEQ ID No. 26):
5′ ACGTTGGATGCTGTAAGTGGTTTCTCAGGA3′
reverse primer (SEQ ID No. 27):
5′ ACGTTGGATGCCAATCATTTAGCTTCACCC3′
extension primer(SEQ ID NO. 28):
5′ AGATCACATTGCAGGGA3′

wherein the presence of C518T polymorphism predicts the poor/intermediate drug metabolism mediated by CYP2C19 enzyme.

In another embodiment, the invention provides an oligonucleotide primer pair suitable for PCR amplifying exon 5 of a CYP2C19 gene having SEQ ID NOS 4 and 5.

In another embodiment, the invention provides oligonucleotide extension primers having sequences selected from the group of polymorphisms represented by positions 518 and consisting of SEQ ID NOS: 12 and 14. In yet another embodiment of the present invention, a method for detecting following polymorphisms CYP2C19*2B, CYP2C19*3 and CYP2C19*5 to identify poor metabolisers, said method comprising the steps of: (a) preparing the nucleic acid template for identifying said polymorphism in said human sample from indian population comprising: (i) isolating double stranded DNA from the human (ii) PCR amplification of DNA in said sample wherein the amplified DNA encodes exons 1, 2, 4, 5 and 9 of CYP2C19 genes present on each homologous chromosome 10 of the human as set forth in SEQ ID NO. 1 (iii) purifying said amplified DNA by polyethylene glycol precipitation (b) incubating a reaction comprising (i) an amount of purified and PCR amplified nucleic acid obtained from said sample sufficient for primer extension, wherein said nucleic acid comprises said P450 2C19 gene sequence, (ii) a nucleic acid polymerase, (iii) a plurality of extension primers that specifically bind to a P450 2C19 gene sequence, and that, when extended by one nucleotide at the 3′ end, comprise a nucleotide indicative of one of a plurality of preselected polymorphisms in said P450 2C19 gene sequence, and (iv) a set of distinctively labeled ddNTPs, under conditions such that at least one of said extension primers is distinctively labeled by addition of one of said ddNTPs comprising a label to the 5′-end of said detection primer, to generate at least one labeled nucleic acid corresponding to at least one of said preselected polymorphisms; and (c) relating the labeled nucleic acid to the identity of said polymorphisms represented by positions 99, 276, 636, 681, 990, 991 and 1297 of SEQ ID NO.1, in said sample.

In another embodiment, the invention provides an oligonucleotide primer pair suitable for PCR amplifying exon 1 of a CYP2C19 gene having SEQ ID NOS 6 and 7.

In yet another embodiment, the invention provides an oligonucleotide primer pair suitable for PCR amplifying exon 2 of a CYP2C19 gene having SEQ ID NOS 8 and 9.

In yet another embodiment, the invention provides an oligonucleotide primer pair suitable for PCR amplifying exon 9 of a CYP2C19 gene having SEQ ID NOS 10 and 11.

In another embodiment, the invention provides oligonucleotide extension primers having sequences selected from the group of polymorphisms represented by positions 99, 276, 636, 990, 991 and 1297 of SEQ ID NO.1, consisting of SEQ ID NOS: 14 through 17 respectively.

Yet, the present invention also provides a kit useful for the prediction of CYP2C19 enzyme mediated drug metabolism comprising:
a) a set of oligonucleotide PCR primers suitable for amplifying the polymorphic region corresponding to position 518 or exon 4 of SEQ ID NO: 2 and 3;
b) a set of sequence determination oligonucleotides for detecting polymorphism at position 518 of SEQ ID NO: 12 and 14;
c) a polymerizing agent and fluorescently labeled chain terminating nucleotides;
d) buffers, vials and microtiter plates.

Further the present invention also provides a method of selecting a dosage of a drug, comprising of adjusting the said dosage compatible to CYP2C19*2C genotype containing the novel polymorphism C518T.

In an embodiment of the present invention, the said drug is a substrate of CYP2C19 enzyme.

In yet another embodiment the invention provides a method of selecting a dosage of a substrate of CYP2C19, said method comprising: selecting said dosage to be compatible with a CYP2C19*2C genotype of said subject identified by the aforementioned method.

By “distinctively labeled”, it is meant that each type of member of a set is labeled with a distinct label that can be distinguished from the other labels. For example, in a set of distinctively labeled nucleotides (e.g., dideoxy NTPs, or ddNPTs), each type of “N” (nucleotide) is labeled with a label that can be distinguished from the other types of labels. Thus, for example, if four labels designated 1, 2, 3, and 4 are used to label the four types of ddNTPs, each ddATP molecule is labeled with label “n”, each ddTTP molecule is labeled with label “2”, each ddCTP molecule is labeled with label “3”, and each ddGTP molecule is labeled with label “*4”. In some aspects of the invention, the distinctive label is a fluorescent label.

As used herein, “primer extension” refers to the enzymatic extension of the three-prime (3′) hydroxy group of an extension primer, which is an oligonucleotide X nucleotides long that is paired to a template nucleic acid (snapshot us pat nio) The extension reaction is catalyzed by a DNA polymerase. By “DNA Polymerase” it is meant a DNA polymerase, or a fragment thereof, that is capable of carrying out primer extension. Thus, a DNA polymerase can be an intact DNA polymerase, a mutant DNA polymerase, an active fragment from a DNA polymerase, such as the Klenow fragment of E. coli DNA polymerase, and a DNA polymerase from any species, including but not limited to thermophilies.

An extension primer has a nucleotide sequence that binds in a complementary fashion to a portion of a sequence of a nucleic acid that encodes or modulates the expression of the cytochrome P450, or to the complement of such a sequence. Preferred extension primers are of a length sufficient to provide specific binding to the sequence of interest. Such primers comprise an exact complement to the sequence of interest for 15 to 40 nucleotides in length, and preferably 20 to 30 nucleotides in length. The extension primer sequence has a 3′ terminus that pairs with a nucleotide base that is, in the sample nucleic acid to which the primer is hybridized, 5′ from the site of one bases in the sequence of interest that represent a polymorphism in a gene.

For example, in the given diagram of a primer extension reaction (FIG. 2), four different ddNTPs, each distinctively labeled, are present in the reaction mixture as designated by dd (A*1)TP, dd (T*2)TP, dd (C*3)TP and dd (G*4)TP, where *1, *2, *3 and *4 represent different labels. In the diagram, the polymorphism in the nucleic acid being tested is indicated by an underlined nucleotide, and the extension primer sequence is italicized. Only one ddNTP, ddTTP, can be added to the 3′ end of the extension primer, because thymine (T) is the only base that pairs with adenosine (A). The addition of the dd (T*2)TP to the 3′ of the primer prevents any further primer extension because it is a dideoxy, chain-terminating ddNTP. Thus, the only primer that is 3′ extended is labeled with label *2. Detection of the signal from label *2 indicates that the A polymorphism is present in the sample.

An amount of nucleic acid sufficient for primer extension can be prepared by amplification via polymerase chain reaction (PCR) using PCR primers. As a non-limiting example, for CYP2C19 appropriate PCR primers include, but are not limited to, those having sequences selected from the group consisting of SEQ ID NOS: 2 through 15.

For each reaction mixture, the amount of the nucleic acid sufficient for primer extension is 0.15 pmol and above and can be determined by obtaining a sample comprising nucleic acid and running it in an appropriate ethidium bromide stained agarose gel together with a DNA marker with different concentrations.

For the purposes of the invention, certain terms are defined as follows “Gene” is defined as the genomic sequence of the CYP2C19 gene.

“Oligonucleotide” means a nucleic acid molecule preferably comprising from about 8 to 50 covalently linked nucleotides. Most preferably, an oligonucleotide of the invention comprises from about 18 to 30 nucleotides.

### “Polymorphisms”

In a normal diploid eukaryote, each gene has 2 loci, i.e., 1 gene copy at the same locus (position) on each of 2 matched chromosomes. Different versions of a gene can occur at any locus, and these versions are called alleles. Alleles include the wildtype (normal) allele and allelic variants.

By “allelic variant” it is meant a variation in a nucleotide sequence, such as a single nucleotide polymorphism (SNP) or any other variant nucleic acid sequence or structure (e.g., duplications, deletions, inversions, insertions, translocations, etc.) in a gene encoding a gene that alters the activity and/or expression of the gene. Allelic variants and/or over- or under-express the polypeptide encoded by the gene, and/or express proteins altered activities by virtue of having amino acid sequences that vary from wild type sequences. Often, more than one allelic variants exist and persist in a population of individuals. By “exist and persist” it is meant that the frequency of incidence of the minor allele(s) is greater than can be explained by recurrent mutation alone (i.e., typically greater than 1%). However, the frequency of any variant allele may vary over time due to such factors as genetic drift and the like. When two or more variant alleles of a gene are present in a population, the gene or the protein it encodes is said to be polymorphic. As used herein, a “polymorphism” refers to a specific allelic variant of a gene or protein.

A “polymorphic” site as defined herein is a portion of a gene that is characterized by at least one polymorphism.

A “genotype” as used herein is a representation of the polymorphic variants present at a polymorphic site.

The nomenclature for human CYP2C19 alleles has been standardized and http://www.imm.ki.se/CYPalleles provides an exemplary list of CYP2C19 alleles.

### Screening for CYP2C19 Polymorphisms:

The present invention provides a novel allelic variant of CYP2C19 gene herein designated CYP2C19*2C and a method for its detection which will be useful in identifying intermediate and poor metabolisers of a substrate of CYP2C19. Specifically in a first step, a nucleic acid is isolated from biological sample obtained from the human. Any nucleic acid containing biological sample from the human is an appropriate source of nucleic acid for use in the methods of the invention. For example, nucleic acid can be isolated from blood, cell scrapings, biopsy tissue, and the like. In the present invention blood sample drawn from the brachial vein served as the source of the genomic DNA for the analyses. DNA is extracted by the salting out procedure⁸. In a second step, using separate PCR reactions sufficient amplicons are generated to assay the polymorphisms of CYP2C19*2C at positions C99T; G276C; C518T; G681A; C990T and A991G of FIG. 1 by the primer extension method described below. For the present invention, to assay the said polymorphisms, five stretches of DNA from the CYP2C19 gene have been amplified using PCR. The DNA stretches which have been amplified correspond to exons 1, 2, 4, 5, 7 and 9 of CYP2C19 gene (GenBank Ref: E10866). The primer sequences used for amplifying each of these exons are: exon 1 is amplified using forward primer SEQ ID NO.6 and reverse primer SEQ ID NO. 7. The corresponding PCR product sequence is provided in SEQ ID NO.18. Exon 2 is amplified using forward primer SEQ ID NO.8 and reverse primer SEQ ID NO. 9. The corresponding PCR product sequence is provided in SEQ ID NO.19. Exon 4 is amplified using forward primer SEQ ID NO. 2 and reverse primer SEQ ID NO. 3. The corresponding PCR product sequence is provided in SEQ ID NO.20. Exon 5 is amplified using forward primer SEQ ID NO.4 and reverse primer SEQ ID NO. 5. The corresponding PCR product sequence is provided in SEQ ID NO:21. Exon 9 is amplified using forward primer SEQ ID NO.10 and reverse primer SEQ ID NO. 11. The corresponding PCR product sequence is provided in SEQ ID NO.22. All the PCR primers were designed to span intron-exon boundaries and the CYP2C19 gene sequence from GenBank Ref: E10866 was used. Oligonucleotides used as PCR primers were obtained from Sigma-Aldrich USA (0.05 micromole scale synthesis, desalted) and stored at minus 20 C under which they are stable for 1 year. PCR-fragments were amplified with Taq DNA polymerase using GeneAmp PCR 9700 (Applied Biosystems).

In a third step, each of the above PCR products is treated, e.g., with polyethylene glycol, to remove excess dNTPs and PCR primers. This is followed by the single nucleotide primer extension SNaPshot reaction. In this reaction, an oligonucleotide primer is designed to have a 3′ end that is one nucleotide 5′ to a specific point mutation site. The primer hybridizes to the PCR amplicon in the presence of fluorescently labeled ddNTPs and a DNA polymerase. The polymerase extends the primer by one nucleotide, adding a single, labeled ddNTP to its 3′ end. Each dideoxynucleotide (e.g., ddATP, ddGTP, ddCTP, ddTTP, ddUTP, etc.) is differently labelled, e.g., each is labeled with a different fluorescent colored dye. Excess ddNTPs are removed from the reaction mixture by calf intestinal phosphatase (CIP) treatment. The products are fluorescently labeled oligonucleotides, each one of which is detected, for example using an automated DNA sequencer (e.g., ABI PRISM 3100 Genetic Analyzer) based on both its size (determined by electrophoretic mobility) and its respective fluorescent label. Based on the presence or absence of polymorphisms at positions 99, 276, 518, 636, 681, 990 and 991 of FIG. 2, polymorphism profile is determined. For the present invention, the SnaPshot primers used to determine polymorphisms at position 99, 276, 518, 636 and 681 correspond to SEQ ID NO. 14; SEQ ID NO. 15; SEQ ID NO. 12; SEQ ID NO. 16; SEQ ID NO. 13 and SEQ ID NO. 17 respectively. The frequencies for each polymorphism was computed in all the participating volunteers and 95% confidence interval calculated.

The invention also provides a diagnostic kit useful for rapid and economical pharmacogenomics based diagnostics for the Indian population. Preferably, the kit of the invention comprises two oligonucleotide primer pairs, wherein each primer pair is complementary to polymorphic regions corresponding to positions 518 of SEQ ID NO 1. The kit may further comprise of sequence determination oligonucleotides for detecting a polymorphic variant corresponding to positions 518 and 681 of SEQ ID NO 1. The kit of the invention may also comprise a polymerizing agent, for example, a thermostable nucleic acid polymerase such as those disclosed in U.S. Pat. Nos. 4,889,818; 6,077,664 and the like. The kit of the invention may also include fluorescently labeled chain terminating nucleotides such as ddATP, ddGTP, ddCTP and ddTTP. The kit of the invention may optionally include buffers, vials, microtiter plates and instructions for use.

In one specific embodiment, the invention provides a kit comprising a pair of oligonucleotide primers (SEQ ID NO 2 & 3) suitable for amplifying the polymorphic region corresponding to position 518 of exon 4 of SEQ ID NO 1, a primer pair (SEQ ID NO 4 & 5) for amplifying the polymorphic region corresponding to position 681 of exon 5 of SEQ ID NO 1, a pair of sequence determination oligonucleotide (SEQ ID NO 12 and SEQ ID No.14) for detecting polymorphism at position 518 of SEQ ID NO 1 and a sequence determination oligonucleotide (SEQ ID NO 13) for detecting polymorphism at position 681 of SEQ ID NO 1.

The invention also provides a method for estimation of drug response associated with the novel allelic variant of the CYP2C19 gene. The method of the study was approved by the institutional ethics committee a priori. The method comprising of selecting volunteers already geno typed to be carrying the CYP2C19*C allele. The selection of volunteers was done after screening them for medical condition judged to influence liver function or requiring pharmacological treatment; any on-going disease, intake of any drug during one week of the study. A written informed consent was obtained from the volunteers. For these experiments, a single oral dose of an appropriate probe drug which is a substrate of CYP2C19 was given to volunteers. The bladder was emptied before drug intake. A single blood sample and urine sample was collected after 4 hours of the study.

### BRIEF DESCRIPTION OF THE DRAWINGS

FIG. 1 represents diagramatic representation of SNaPshot reaction.

FIG. 2 represents the sequence of the CYP2C19 gene (GenBank sequence accession: E10866) with polymorphic sites underlined and highlighted in bold and underlined.

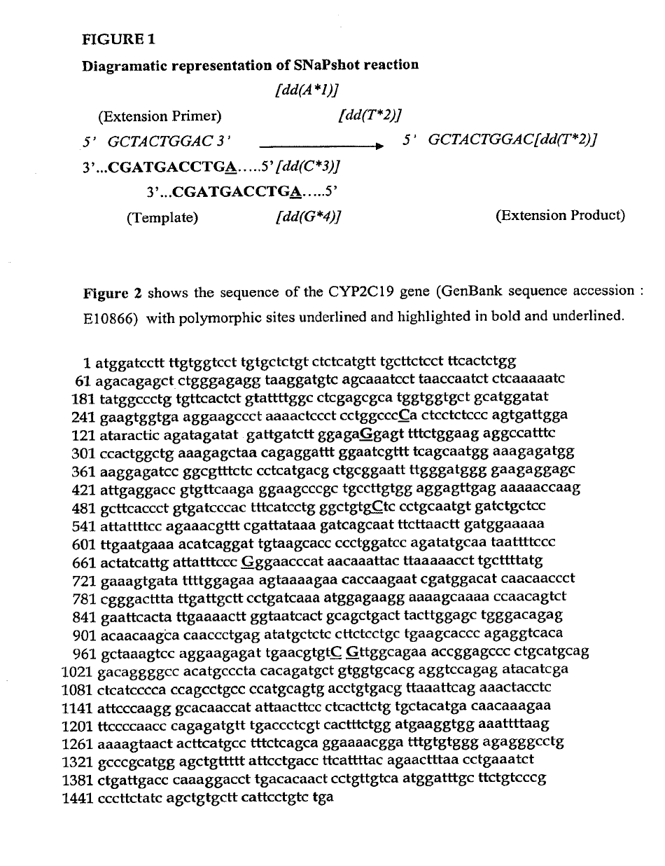

The following are given by way of illustration only and therefore, should not be construed to limit the scope of the present invention.

### EXAMPLES

### Example 1

### I. Identification of Novel Polymorphism in CYP2C19 Gene

The inventors have identified novel polymorphic site in the Indian population in a contiguous region of the coding sequence of the exon 4 of CYP2C19 gene in Indian population.

### Example 2

### II. Single Polymorphism of the Invention as a Poor and Intermediate Drug Metabolism

The applicants carried out the PCR amplification of 4^{th} exonic region of the human CYP2C19 gene using oligonucleotide primers. These primers were designed in accordance with the human CYP2C19 gene sequence submitted by DOE Joint Genome Institute and Stanford Human Genome Center (6 Oct. 1999) (GenBank accession number-E10866). The sequencing of the purified PCR product revealed heterozygous nonsynonymous polymorphism in 4th exonic region of the human CYP2C19 gene associated with poor and intermediate drug metabolism. The present invention provides a sequence for the allelic variants of human CYP2C19 gene comprising nonsynonymous polymorphism in 4^{th} exonic region of the human CYP2C19 gene sequence in the database (GenBank Accession No.-E10866) associated with drug metabolism.

TABLE 1

| Site of change | Base change | Amino-acid alteration |
|---|---|---|
| 518 | C→ T | Ala 173Val |

The sites of changes are in accordance with the PCR Product Sequence obtained using primers (SEQ ID 2 and 3) flanking 4^{th} exonic region of the human CYP2C19 gene (GenBank accession number-E10866).

The substitution C→T changes amino acid Ala173Val which consequently leads to the nucleotide sequence of the allelic variant of exonic region of the human CYP2C19 gene. PCR Product Sequence containing the nonsynonymous polymorphism is obtained using primers SEQ ID 2 and 3 flanking nonsynonymous polymorphism in 4^{th} exonic region of the human CYP2C19 gene of SEQ ID 1.

The polymorphic site is at nucleotide position 518 in the above sequence (C*) the primers are used to detect polymorphism at position 518 according to the PCR product obtained using primers (SEQ ID 2 and 3) flanking 4^{th} exonic region of the human CYP2C19 gene (Table 1).

### Example 3

### V) Diagnostic Kits

The invention further provides a diagnostic kit for predicting an individual's response to a beta agonist comprising:
PCR amplification primers of SEQ ID 2 and 3,
Snapshot primer of SEQ ID No. 4 or 5,
At least one allele-specific oligonucleotide selected from oligonucleotides of SEQ ID Nos. 6, 7, 8, and 9.
Appropriate buffers for PCR or hybridization reactions.

The allele-specific oligonucleotides may alternatively be provided as immobilized to a substrate, which can be used to detect polymorphism in CYP2C19 gene. Optional additional components of the kit include, for example, restriction enzymes, polymerase, the substrate nucleoside triphosphates, means used to label (for example, an avidin enzyme conjugate and enzyme substrate and chromogen if the label is biotin).

### Example 4

### Identification of Polymorphisms in CYP2C19 Gene

The inventors identified 6 polymorphic sites in a contiguous region of the coding sequence of the CYP2C19 gene in Indian population (Table2). It illustrates examination of the six polymorphic sites from the coding region of the CYP2C19 gene.

Exons 1, 2, 4, 5, 7 and 9 were amplified from genomic DNA from the normal Indian individuals using the following PCR primers, with the indicated positions corresponding to GenBank Accession No. E 10866. The identified polymorphisms are shown in Table 2 and FIG. 2.

TABLE 2

| Site of change | Base change |
|---|---|
| 99 | C→ T |
| 276 | G→ C |
| 518 | C→ T |
| 681 | G→ A |
| 990 | C→ T |
| 991 | A→ G |

### Example 5

### Identification of Nonsynonymous Polymorphism in CYP2C19 Gene

This example describes the identification of nonsynonymous polymorphism in exonic region of CYP2C19 gene by PCR and sequencing, using certain oligonucleotide primers according to the invention.

Genomic DNA was isolated from peripheral blood using salt-precipitation method (Miller et al., 1988). The concentration of the DNA was determined by measuring the absorbance of the sample, at a wavelength of 260 nm. The DNA from asthmatics was then amplified by polymerase chain reaction by using the oligonucleotide primer 2 and 3 (SEQ ID 2 and 3). Each 500 PCR reaction contained 200 ng DNA, 20 pmol each of oligonucleotide primer 2 and 3 (SEQ ID 2 and 3), 1.8 units Taq Polymerase (Bangalore Genei), and 200 mM deoxyribonucleoside triphosphate (dNTP) in a 10×PCR buffer (containing 100 mM Tris (pH 9.0), 500 mM KCl, and 0.1% Gelatin).

The samples were denatured at 94° C. for 5 min followed by 37 cycles of denaturation 94° C., 45 sec), annealing (56° C., 1 min), extension (72° C., 1.2 min) and a final extension of 10 min at 72° C. in a Perkin Elmer Gene Amp PCR System 9600. This reaction produced a DNA fragment of 954 bp. PCR products were purified by Poly Ethylene Glycol/Sodium acetate solution (containing PEG 8000, 1M Magnesium chloride and 3M anhydrous Sodium acetate, pH-4.8) and both the strands of the PCR product were directly sequenced using dye terminator chemistry on an ABI Prism 3100 automated DNA sequencer. The PCR product was shown to be identical to the exon of the CYP2C19 gene sequence in the database (Accession Number-E 10866). Sequences were aligned with the corresponding wild-type sequences using the Factura and Sequence Navigator software programs

### Example 6

### Screening Polymorphism in the Population

This example describes a primer extension reaction used to screen single nucleotide variants. The DNA samples from several human subjects were amplified by PCR and the PCR products were purified as described in example 5. The primer extension reaction was performed on the purified PCR products using oligonucleotide primer and SNaPshot ddNTP primer extension kit (PE Biosystems). The snapshot technique is extensively used in the molecular studies and is useful in exact base identity determination of a polymorphic locus. Although, the basic methodology followed for all snapshot protocols is same in all studies. But the each snapshot protocol is unique in itself. This is because each protocol is locus specific. Therefore, a specific working protocol has to be developed and invented for identification of specific locus. In other words the reaction and PCR conditions developed using the snapshot technique in the present study is different from any other snapshot technique used for any other disease locus. This means that the novel specific protocol of snapshot technique as given in the present invention has been established for this very specific locus i.e for CYP2C19 locus. This protocol will only work if only these specific designed and developed primers having SEQ ID No. 4 and SEQ ID No.5 are used. The oligonucleotide primer was designed till the penultimate position of mutation and the primer is extended by one ddNTP, which is in accordance with the variant allele present. The reaction was performed for 30 cycles of denaturation (96° C., 10 sec), annealing (55° C., 5 sec) and extension (60° C., 30 sec) in a Perkin Elmer GeneAmp PCR System 9600 using primers having SEQ ID No. 4 and SEQ ID No.5. The primer extension products were treated with calf intestine alkaline phosphatase (New England Biolabs) for removing unincorporated dideoxynucleotides. The products were run on an ABI Prism 3100 automated DNA sequencer. Depending on the colour of the fluoroscently labeled dideoxynucleotide incorporated, the wild type and polymorphic alleles of the CYP2C19 gene were detected. Results are given in table 3.

### Example 7

### Nucleotide Sequence of Allelic Variants of CYP2C19 Gene (Accession Number E10866)

The nucleotide sequence of the allelic variants of CYP2C19 gene derived using the method as described in example 5 for six polymorphic locations including novel site of the invention:

| | |
|---|---|
| 1 | atggatcctt ttgtggtcct tgtgctctgt ctctcatgtt tgcttctcct ttcactctgg |
| 61 | agacagagct ctgggagagg aaaactccct cctggcccCa ctcctctccc agtgattgga |
| 121 | aatatcctac agatagatat taaggatgtc agcaaatcct taaccaatct ctcaaaaatc |
| 181 | tatggccctg tgttcactct gtattttggc ctcgagcgca tggtggtgct gcatggatat |
| 241 | gaagtggtga aggaagccct gattgatctt ggagaGgagt tttctggaag aggccatttc |
| 301 | ccactggctg aaagagctaa cagaggattt ggaatcgttt tcagcaatgg aaagagatgg |
| 361 | aaggagatcc ggcgtttctc cctcatgacg ctgcggaatt ttgggatggg gaagaggagc |
| 421 | attgaggacc gtgttcaaga ggaagcccgc tgccttgtgg aggagttgag aaaaaccaag |
| 481 | gcttcaccct gtgatcccac tttcatcctg ggctgtgCtc cctgcaatgt gatctgctcc |
| 541 | attattttcc agaaacgttt cgattataaa gatcagcaat ttcttaactt gatggaaaaa |
| 601 | ttgaatgaaa acatcaggat tgtaagcacc ccctggatcc agatatgcaa taattttccc |
| 661 | actatcattg attatttccc Gggaacccat aacaaattac ttaaaaacct tgcttttatg |
| 721 | gaaagtgata ttttggagaa agtaaaagaa caccaagaat cgatggacat caacaaccct |
| 781 | cgggacttta ttgattgctt cctgatcaaa atggagaagg aaaagcaaaa ccaacagtct |
| 841 | gaattcacta ttgaaaactt ggtaatcact gcagctgact tacttggagc tgggacagag |
| 901 | acaacaagca caaccctgag atatgctctc cttctcctgc tgaagcaccc agaggtcaca |
| 961 | gctaaagtcc aggaagagat tgaacgtgtC Gttggcagaa accggagccc ctgcatgcag |
| 1021 | gacaggggcc acatgcccta cacagatgct gtggtgcacg aggtccagag atacatcga |
| 1081 | ctcatcccca ccagcctgcc ccatgcagtg acctgtgacg ttaaattcag aaactacctc |
| 1141 | attcccaagg gcacaaccat attaacttcc ctcacttctg tgctacatga caacaaagaa |
| 1201 | ttccccaacc cagagatgtt tgaccctcgt cactttctgg atgaaggtgg aaattttaag |
| 1261 | aaaagtaact acttcatgcc tttctcagca ggaaaacgga tttgtgtggg agagggcctg |
| 1321 | gcccgcatgg agctgttttt attcctgacc ttcattttac agaactttaa cctgaaatct |
| 1381 | ctgattgacc caaaggacct tgacacaact cctgttgtca atggatttgc ttctgtcccg |
| 1441 | cccttctatc agctgtgctt cattcctgtc tga |

Exon1: 1-168
Exon2: 168-331
Exon3: 332-481
Exon4: 483-642
Exon5: 643-820
Exon6: 821-961
Exon7: 962-1149
Exon8: 1150-1291
Exon9: 1292-1473

### Example 8

### The Association of Non-Synonymous Polymorphism with Drug Metabolism

The non-synonymous SNP or polymorphism are defined as “when the altered code doesn't correspond to the same amino acid as the wild type sequence i.e these are substitutions in coding region that result in a different amino acid”.

Frequency of the C and T alleles at position 518 in the populations from different geographical regions of India and belonging to different linguistic lineages (Indo-European and Austro-Asiatic) and from large, isolated as well as admixed population were analysed. The average frequency of the minor allele ‘T’ was found to vary from 0.8% to 15% (Table 3 and 4). Overall the average frequency in the Indian population is about 14%. (Table 4).

TABLE 3

| Code | | Frequency | C518T | C518C |
|---|---|---|---|---|
| IE-E-LP1 | east | 42 | 0.10 | 0.90 |
| IE-E-LP2 | east | 30 | 0.13 | 0.87 |
| IE-N-SP1 | North | 40 | 0.05 | 0.95 |
| IE-N-SP1 | north | 44 | 0.05 | 0.95 |
| IE-N-IP1 | North | 46 | 0.07 | 0.93 |
| IE-N-LP9 | North | 46 | 0.09 | 0.91 |
| IE-N-LP8 | north | 36 | 0.17 | 0.83 |
| IE-N-LP10 | north | 38 | 0.08 | 0.92 |
| TB-N-IP1 | north | 46 | 0.09 | 0.91 |
| IE-S-IP1 | south | 46 | 0.15 | 0.85 |
| IE-S-IP1 | south | 42 | 0.14 | 0.86 |
| IE-W-IP1 | West | 36 | 0.11 | 0.89 |
| IE-W-LP1 | West | 46 | 0.00 | 1.00 |
| AA-W-IP1 | West | 38 | 0.11 | 0.89 |
| AA-W-IP1 | West | 44 | 0.11 | 0.89 |
| IE-W-LP2 | West | 46 | 0.00 | 1.00 |
| IE-W-LP3 | West | 46 | 0.17 | 0.83 |
| IE-W-LP3 | West | 44 | 0.09 | 0.91 |
| IE-N-SP1 | North | 40 | 0.05 | 0.95 |
| IE-N-SP1 | north | 44 | 0.05 | 0.95 |
| IE-N-IP1 | North | 46 | 0.07 | 0.93 |
| IE-N-LP9 | North | 46 | 0.09 | 0.91 |
| IE-N-LP8 | north | 36 | 0.17 | 0.83 |
| IE-N-LP10 | north | 38 | 0.08 | 0.92 |
| TB-N-IP1 | north | 46 | 0.09 | 0.91 |
| IE-E-LP1 | east | 42 | 0.10 | 0.90 |
| IE-E-LP2 | east | 30 | 0.13 | 0.87 |
| IE-S-IP1 | south | 46 | 0.15 | 0.85 |
| IE-S-IP1 | south | 42 | 0.14 | 0.86 |
| IE-W-IP1 | West | 36 | 0.11 | 0.89 |
| IE-W-LP1 | West | 46 | 0.00 | 1.00 |
| AA-W-IP1 | West | 38 | 0.11 | 0.89 |
| AA-W-IP1 | West | 44 | 0.11 | 0.89 |
| IE-W-LP2 | West | 46 | 0.00 | 1.00 |
| IE-W-LP3 | West | 46 | 0.17 | 0.83 |
| IE-W-LP3 | West | 44 | 0.09 | 0.91 |

TABLE 4

| Geographical | No of | frequency of | frequency of |
|---|---|---|---|
| Zone | samples | T allele (%) | C allele (%) |
| North India | 296 | 8.0 | 92 |
| Western states | 300 | 2.0 | 92 |
| of India | | | |
| Eastern states | 72 | 11.0 | 89 |
| of India | | | |
| South India | 88 | 15.0 | 85 |
| Total | 756 | 14.0 | 89 |
| frequency | | | |

Genotypes
CC - 322
CT - 67
TT - 2

The genotypes CC, CT and TT have been found to be in Hardy Weinberg equilibrium and are stabilized in all the subpopulations.

HWEχ²=0.5615, p=0.45

The data clearly shows that the average frequency of the allele T and allele C in the Indian population from various ethnic groups and geographical regions is 14% and 89% respectively (Table 4).

The distribution and frequencies of CYP2C19 polymorphisms differ widely among different populations and ethnic groups, and association studies have established concomitant differences in CYP2C19 activity and responses to drugs which are CYP2C19 substrates. In Indian population subjects mainly from North India showed PM frequency of 11% ⁷. The PM condition is inherited as an autosomal recessive trait⁴. Similar frequency of the novel variant C518T in the Indian population suggests that this variant could be associated with poor metabolizing effect.

The best characterized defect due to CYP2C19 polymorphisms responsible for the PM phenotype are a single base pair substitution in exon 5 at position 681 (G→A) of the coding sequence (GenBank Ref NM_{—}000769),⁵. The change in CYP2C19*2 creates an aberrant splice site, resulting in truncated, inactive protein. This polymorphism accounts for 75% of the defective alleles in orientals and 93% in Caucasians. The other well characterized detrimental allele CYP2C19*3 discovered in Japanese PMs, results in a stop codon and consequently an inactive protein. This allele accounts for approximately 25% of all inactive forms in orientals, being by converse extremely rare in non-oriental populations. Intermediate metabolisers can be predicted from identification of heterozygous carriers of one deficient allele of CYP2C19*2 and CYP2C19*3 and the other CYP2C19*1 allele. C518T appears to be a unique polymorphism as it has been found to exist in heterozygous and can function as poor/intermediate metaboliser. An individual having CC genotype is expected to be a normal metaboliser and one with CT genotype is expected to be a intermediate metaboliser.

### Example 9

This example describes a method of high throughput analysis for novel SNP detection by sequenome. The system provides a unique combination of mass spectrometry and a biochemical reaction that extends a short primer through the region of novel SNP. The system allows unprecedented accuracy since the molecular masses of the diagnostic products are measured directly. The power of SEQUENOM's MassARRAY technology resides in its ability to rapidly distinguish genotypes with a high level of precision and sensitivity. Using MALDI-TOF mass spectrometry, DNA fragments associated with novel genetic variants were validated in the various Indian populations.

This protocol will only work if the specific designed and developed primers having SEQ ID No. 25, 26 and 27 are used. The PCR reaction was performed at denaturation (95° C., 20 sec), annealing (56° C. 30 sec) and extension (72° C., 60 sec) for 45 cycles in a Perkin Elmer GeneAmp PCR System 9600 using primers having SEQ ID No. 25 and SEQ ID No.26. The PCR products were treated with shrimp alkaline phosphate. The amplicons were extended by hME (homogenous mass extend) primer SEQ ID 27. To the products of the hME reaction resin treatment were given for 30 min before running in Sequenome. Depending on the charge and mass ratio of the product, the wild type and polymorphic alleles of the CYP2C19 gene were detected.

### Example 10

### The Method for Estimation of Drug Response

The invention also provides a method for estimation of drug response associated with the novel allelic variant of the CYP2C19 gene. The method of the study was approved by the institutional ethics committee a priori wherein the volunteers are genotyped to be carrying the CYP2C19*C allele. The selection of volunteers is done after screening them for medical condition judged to influence liver function or requiring pharmacological treatment; any on-going disease, intake of any drug during one week of the study. A written informed consent is obtained from the volunteers. For these experiments, a single oral dose of an appropriate probe drug, which is a substrate of CYP2C19 is given to volunteers. The bladder is emptied before drug intake. A single blood sample and urine sample is collected after 4 hours of the study.

### REFERENCES

1. Pharmacogenomics: implications for laboratory medicine, Clinica Chimica Acta 308:43-53, 2001
2. Linder et al., Pharmacogenetics: a laboratory tool for optimizing therapeutic efficiency, Clinical Chemistry 43:2, 254-266, 1997;
3. Kalow, Pharmacogenetics in Biological Perspective, The American Society for Pharmacology and Experimental Therapeutics, 49:0369-0379, 1997.
4. De Morais et al., Identification of a new genetic defect responsible for the polymorphism of (S)-mephenyloin metabolism in Japanese. Mol. Pharmacol. 46:4, 594-598, 1994.
5. De Morais et. al, The major genetic defect responsible for the polymorphism of S-mephenyloin metabolism in humans. J Biol Chem 269:22, 15419-15422, 1994.
6. Ibeanu et al, Identification of new human CYP2C19 alleles (CYP2C19*6 and CYP2C19*2B) in a Caucasian poor metabolizer of mephenyloin. J Pharmacol Exp Ther. 286: 31490-31495, 1998.
7. Lamba et. al. CYP2C19 genetic mutations in North Indians. Clin Pharmacol Ther 60: 5, 138-144, 2000.
8. Miller et. al. Nucleic Acids Research 16: 3, 1215, 1988.
9. Jungerius et. al. Typing single-nucleotide polymorphisms using a gel-based sequencer: a new data analysis tool and suggestions for improved efficiency. Mol. Biotechnol. 25:3, 283-8, 2003.
10. Lamba V, Lamba J K, Dilawari J B, Kohli K K. Genetic polymorphism of CYP2D6 in North Indian subjects. Eur J Clin Pharmacol 1998; 54:787-91.
11. Lamba J K et al (1998). Genetic polymorphim of the hepatic cytochrome p450 2C19 in North Indian subjects. Clinical Pharmacology and therapeutics, Vol. 63, pages 422-427
11. Nowak M P, Seller E M, and Tyndale R F (1998). Canadian Native Indians exhibit unique CTP2A6 and CTP2C19 mutant allele frequencies. Clinical Pharmacology and therapeutics, Vol. 64, pages 378-383
12. C. Adithan, N. Gerard, S. Vasu, J. Rosemary, C. H. Shashindran & R. Krishnamoorthy, 2003 J Clin Pharmacol 56, 331-333
13. Lamba J K, Dhiman R K, Singh R and Kohli K K (2001) Eur. J. Clin. Pharmacol. 57; 649-652.
14. Badyal D K and Dadhuch, A P (2001), Indian J. Pharmacol. 33; 248-259.
15. Desai C A and Smanat B D (2002), Drug Review, 48; 80-81.
16. Gaedigk, A (2000) Intl. J. Clinical Pharmacol. Therapeutics, 38; 61-68.

1-8. (canceled)

9. A method for predicting the capacity of a drug dose to metabolize a substrate of a CYP2C19 enzyme in a human subject from indian population, wherein the said method comprising the steps of (a) preparing the nucleic acid template for identifying the polymorphism at positions 518 of SEQ ID NO: 2 and 3 comprising:
(i) isolating double stranded DNA from the subject;
(ii) PCR amplification of the DNA wherein the amplified DNA encodes exon 4 of CYP2C19 gene present on each homologous chromosome 10 of the subject as set forth in SEQ ID NO. 2 and 3 respectively;
(iii) purifying said amplified DNA by polyethylene glycol precipitation;
(b) detecting polymorphism using extension primers (Snap short primers) having SEQ ID No. 12 and 14;
(c) relating the labeled nucleic acid to the identity of the said polymorphism in the subject and validating the polymorphism by high throughput mass array detector using PCR primers comprising SEQ ID No. 26 and SEQ ID No. 27 and extension primer having SEQ ID 28, wherein:
forward primer (SEQ ID No. 26):
5′ ACGTTGGATGCTGTAAGTGGTTTCTCAGGA3′
reverse primer (SEQ ID No. 27):
5′ ACGTTGGATGCCAATCATTTAGCTTCACCC3′
extension primer(SEQ ID NO. 28):
5′ AGATCACATTGCAGGGA3′
wherein the presence of C518T polymorphism predicts the poor/intermediate drug metabolism mediated by CYP2C19 enzyme.

10. A method as claimed in claim 9, wherein the patient is undergoing treatment for any disease with a drug that is metabolized by CYP2C19 enzyme, wherein the said drug is selected from a group of proton pump inhibitors comprising omeprazole, lansoprazole and pentaprazole; antimalarial drugs such as proguanil; antidepressants such as citalopram; the benzodiazepines diazepam and flunitrazepam.

11. A method as claimed in claim 9, wherein average frequency of allele T and C of CYP2C19 gene in the Indian population from the various ethnic populations is 14% and 89% respectively.

12. A kit useful for the prediction of CYP2C19 enzyme mediated drug metabolism comprising:
a) a set of oligonucleotide PCR primers suitable for amplifying the polymorphic region corresponding to position 518 or exon 4 of SEQ ID NO: 2 and 3;
b) a set of sequence determination oligonucleotides for detecting polymorphism at position 518 of SEQ ID NO: 12 and 14;
c) a polymerizing agent and fluorescently labeled chain terminating nucleotides;
d) buffers, vials and microtiter plates.

13. A method of selecting a dosage of a drug, comprising of adjusting the said dosage compatible to CYP2C19*2C genotype containing the novel polymorphism C518T identified by the method of claim 9.

14. The method of claim 12, wherein the said drug is a substrate of CYP2C19 enzyme.

15. A method comprising use of novel allelic variant having SEQ ID No.24 CYP2C19*2C of CYP2C19 gene for the prediction of CYP2C19 enzyme mediated drug metabolism.
